(19) United States
(12) Patent Application Publication
(10) Pub. No.: US 2026/0041921 A1
(43) Pub. Date: Feb. 12, 2026

(54) MULTI-WIRELESS COMMUNICATION-BASED NEUROSTIMULATION ELECTROCEUTICAL WITH INTEGRATED POWER HARVESTING UNIT

(71) Applicant: ENERGY MINING Co., Ltd., Suwon-si (KR)

(72) Inventors: Hyunmoon PARK, Gunpo-si (KR); Changheon LEE, Suwon-si (KR); Joonha HWANG, Suwon-si (KR)

(73) Assignee: ENERGY MINING Co., Ltd., Suwon-si (KR)

(21) Appl. No.: 18/964,028

(22) Filed: Nov. 29, 2024

(30) Foreign Application Priority Data
Aug. 9, 2024 (KR) 10-2024-0106543
Aug. 9, 2024 (KR) 10-2024-0106546
Aug. 26, 2024 (KR) 10-2024-0114225

(51) Int. Cl. A61N 1/378; A61N 1/02; A61N 1/36; A61N 1/372; A61N 1/375; H02N 1/04

(52) U.S. Cl. CPC A61N 1/3787; A61N 1/025; A61N 1/36175; A61N 1/37217; A61N 1/375; H02N 1/04

(57) ABSTRACT

The present disclosure provides an electroceutical including: a housing including a plurality of regions; a triboelectric power harvesting unit configured to generate energy based on an ultrasound wave provided from outside of the electroceutical; a plurality of antennas operating in different frequency bands; and shielding metal configured to shield electromagnetic interference by at least one of the plurality of antennas.

### CROSS-REFERENCE TO RELATED PATENT APPLICATION

This application claims the benefit of Korean Patent Application Nos. 10-2024-0114225, filed on Aug. 26, 2024, 10-2024-0106543, filed on Aug. 9, 2024, and 10-2024-0106546, filed on Aug. 9, 2024, in the Korean Intellectual Property Office, the disclosures of which are incorporated herein in their entirety by reference.

### BACKGROUND OF THE DISCLOSURE

### Field of the Disclosure

The present disclosure relates to a multi-wireless communication-based neurostimulation electroceutical with an integrated power harvesting unit, and more particularly, to a neurostimulation electroceutical including a plurality of antennas operating in different frequency bands and a power harvesting unit.

### Background of the Related Art

With an increasing demand for maintaining a healthy life in aging societies, a demand for implantable medical devices has grown rapidly since 2010, especially in advanced countries. In particular, along with advancements in state-of-the-art IT technology, the implantable medical devices become smaller, exhibit high performance, and require low power. Owing to a size reduction to a few centimeters, the implantable medical devices are now referred to as electroceuticals. However, since operation of the implantable medical devices still relies on batteries to operate like in 1970s, these devices need to be replaced due to battery depletion.

Thus, to minimize side effects of surgeries due to replacement of implantable medical devices and economic and psychological burdens caused by the surgeries, research into methods of recharging implantable devices and low-power technology is being conducted. Until the early 2000s, alternating current (AC) signals were used to deliver stimulation. AC signaling is a traditional method of stimulating nerves through signals with particular frequencies and voltages. However, this method consumes a lot of energy, and has a limitation in a device size and battery life.

With advancements in medical and semiconductor technologies, a method of controlling rectified direct current (DC)-based neural stimulation has been developed. This method enables precise control in units of micrometers (μm) and provides energy efficiency twice or more than that of an alternating current (AC) method. By doing so, low power characteristics and miniaturization of a device may be implemented, thus allowing for design of multi-electrode arrays attachable to peripheral nerves of patients for effective treatment of patients with various diseases including Alzheimer's disease (AD), peripheral neuropathy, diabetic neuropathy, and posttraumatic pain. In addition, research is also being conducted to enable personalized treatment such that patients may adjust stimulation intensity, frequencies, and pulses through an external programmer. However, a closed-loop nerve stimulation method of providing stimulation by indirectly measuring electromyography, a heart rate, blood pressure, etc. comprehensively has been inefficient.

However, when real-time sensing of a target nerve with respect to stimulation may be performed, monitoring only electroencephalography (EEG) for vagus nerves or only a heart rate for peripheral nerves may allow continuous monitoring and optimization of an influence of nerve stimulation on an entire body, and an effect of the nerve stimulation. Thus, an active and efficient treatment may be performed.

Since there had been a difficulty in existing peripheral nerve stimulation, peripheral nerve stimulation devices for hands or feet in a form of external stimulators were introduced. However, these peripheral nerve stimulation devices could not be widely used due to low efficiency. In addition, since maximum efficiency of the nerve stimulators, i.e., an ‘immediate effect’ was not be provided, these peripheral nerve stimulation devices merely alleviate symptoms through repetitive and periodic treatment.

In addition, due to miniaturization of implantable nerve stimulators and limitations in battery supply and usability, other leading overseas companies are providing miniaturized nerve stimulators that may be powered externally through wireless technology and wireless charging, and thus, do not need batteries. However, such miniaturized nerve stimulators employ passive rather than active methods, and thus, may only perform sensing based on monitoring before implantation. Accordingly, there are limitations in monitoring and correcting a condition of a patient in real time after implantation.

### SUMMARY OF THE DISCLOSURE

Therefore, the present disclosure has been made to solve the above-described problems, and it is an object of the present disclosure to provide an electroceutical that does not need a battery or battery replacement. The electroceutical in the present disclosure capable of being operated or controlled using different radio frequencies may be powered through ultrasound waves in a low-frequency ultrasound (LFU) range or a low-intensity pulsed ultrasound (LIPUS) range based on an integrated triboelectric power harvesting unit, and provide nerve stimulation and sensing through an analog-to-digital converter (ADC) and pulse width modulation (PWM). However, this is only an example, and the scope of the present disclosure is not limited thereto.

To accomplish the above object, according to one aspect of the present disclosure, there is provided an electroceutical including: a housing including a plurality of regions; a triboelectric power harvesting unit configured to generate energy based on an ultrasound wave provided from outside of the electroceutical; a plurality of antennas operating in different frequency bands; and shielding metal configured to shield electromagnetic interference by at least one of the plurality of antennas.

According to one example, the plurality of antennas may include: a first antenna configured to transceive a power control signal for the electroceutical through a near field communication (NFC) band; a second antenna having an operating frequency of 2.4 GHz; and a third antenna configured for a medical implant communication service (MICS).

According to one example, the plurality of regions in the housing may include: a first silicon region provided to cover at least a portion of the first antenna; a second silicon region provided to cover the second antenna and the third antenna; and a titanium region located between the first silicon region and the second silicon region.

According to one example, the electroceutical may further include a printed circuit board (PCB) substrate, wherein the plurality of antennas and the shielding metal are arranged on one surface of the PCB substrate, and the triboelectric power harvesting unit is attached to an inner surface of the titanium region and is spaced apart from the PCB substrate.

According to one example, the electroceutical may further include a header connector arranged on the one surface of the PCB substrate, wherein the first antenna is arranged further adjacent to the header connector compared to the second antenna and the third antenna, and the shielding metal extends in a longitudinal direction of the first antenna to be arranged between the first antenna and the header connector.

According to one example, the electroceutical may further include a microcontroller unit (MCU) configured to change a setting of the header connector according to an operation mode, wherein, based on a control signal from an external electronic device, the MCU integrates and manages a function of an analog-to-digital converter (ADC) configured to sense an analog signal generated from a nerve and a function of a pulse width modulation (PWM) configured to provide a stimulation to the nerve.

According to one example, the electroceutical may further include an ADC configured to convert alternating current (AC) triboelectricity generated by the triboelectric power harvesting unit into direct current (DC) triboelectricity; and a storage configured to store the DC triboelectricity obtained as a result of the converting.

According to one example, the triboelectric power harvesting unit may include: a power generating unit including a first unit, a second unit, and a third unit; a first silicon layer arranged between an upper surface of the power generating unit and an inner surface of the electroceutical; a second silicon layer arranged on a lower surface of the power generating unit; and a device housing arranged to surround outer peripheries of the power generating unit, the first silicon layer, and the second silicon layer.

According to one example, the ultrasound wave provided from the outside may penetrate through a titanium region and the first silicon layer to be introduced into the power generating unit, be reflected within the power generating unit, and cause repetitive vibrations.

According to one example, the device housing and the shielding metal include mu-metal including at least one among nickel, iron, copper, and molybdenum.

In addition to those described above, other aspects, features and effects will become apparent from the following drawings, claims, and detailed descriptions of the present disclosure.

As described above, according to one embodiment of the present disclosure, the present disclosure may minimize battery replacement in an electroceutical utilizing ultrasound waves in a medical frequency range, and efficiently perform low-power control of an implantable electroceutical through utilization of various communication frequencies that may be covered by the electroceutical. However, the scope of the present disclosure is not limited by the effects described above.

### BRIEF DESCRIPTION OF THE DRAWINGS

The above and other objects, features and advantages of the present disclosure will be apparent from the following detailed description of the embodiments of the disclosure in conjunction with the accompanying drawings, in which:

FIGS. 1 and 2 are diagrams for explaining an electroceutical;

FIG. 3A is a front view and FIG. 3B is a rear view of the electroceutical;

FIG. 4 is a front cross-sectional view of the electroceutical;

FIG. 5 is a diagram for explaining a medical implant communication service (MICS);

FIGS. 6A-6C and FIG. 7 are diagrams for explaining noise filtering; and

FIGS. 8 and 9 are diagrams for explaining a triboelectric power harvesting unit.

### DETAILED DESCRIPTION OF THE PREFERRED EMBODIMENT

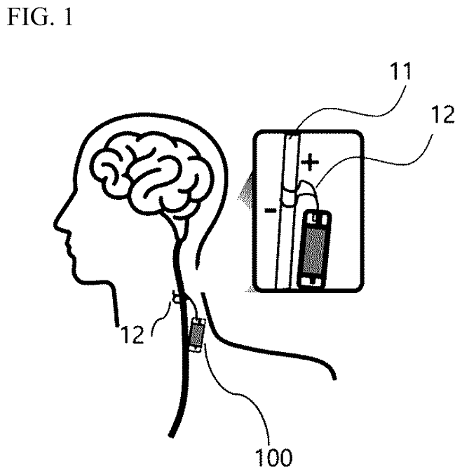

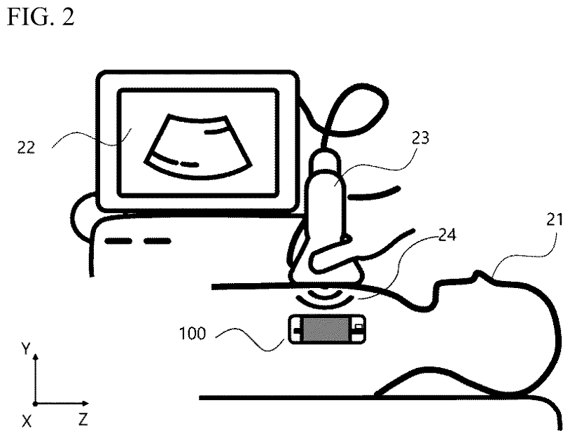

FIGS. 1 and 2 are diagrams for explaining an electroceutical.

Referring to FIG. 1, a position and operation of an electroceutical 100 are shown. The electroceutical 100 (e.g., an electronic device) may refer to a medical device implanted into a particular portion inside a body to stimulate a nerve 11 or sense biometric data. The electroceutical 100 is an implantable medical device and may be located at a depth of 2 cm or more inside the body. The electroceutical 100 is located deep in the body to be protected from an external impact or an environmental change.

The electroceutical 100 may be connected to the nerve 11 through a cuff lead 12. The electroceutical 100 may control or improve a function of each organ provided through an electric signal provided through the cuff lead 12. The electroceutical 100 may include a battery therein for long-term operation.

Referring to FIG. 2, a schematic diagram related to charging of the electroceutical 100 is shown. The electroceutical 100 is a medical device implanted into a body of a person 21, and thus, it may not be easy to replace a battery. Therefore, the electroceutical 100 inside the body may charge a battery based on an ultrasound wave 24 provided from outside of the body.

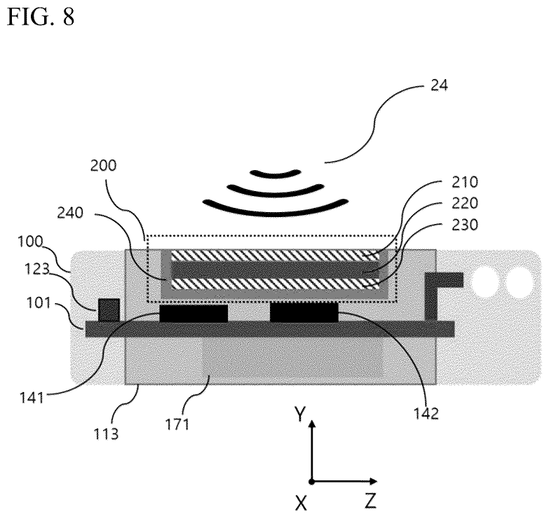

First, an ultrasound monitoring device 22 may be utilized to identify a position of the electroceutical 100 implanted into the body of the person 21. When the position of the electroceutical 100 is identified through the ultrasound monitoring device 22, the ultrasound wave 24 may be provided into the body through an ultrasound probe 23 (e.g., an ultrasound generator). The ultrasound wave 24 may penetrate through the body to be introduced into the electroceutical 100. The ultrasound wave 24 introduced into the electroceutical 100 may vibrate a triboelectric power harvesting unit (e.g., a triboelectric power harvesting unit 200 of FIG. 8) of the electroceutical 100. The vibration may generate electric charges within the triboelectric power harvesting unit, and the electroceutical 100 may be charged through the generated electric charges.

The electroceutical 100 with an approximately rectangular shape may be located such that two widest surfaces (e.g., surfaces parallel to an XZ plane) are parallel with an elongation direction (e.g., a Z-axis direction) of the person 21. Among the two widest surfaces, a surface located at a shallowest portion of the body may be defined as a front surface, and a surface located at a deepest portion thereof may be defined as a rear surface. The ultrasound wave 24 may penetrate through the body to be introduced into the electroceutical 100 through the front surface of the electroceutical 100.

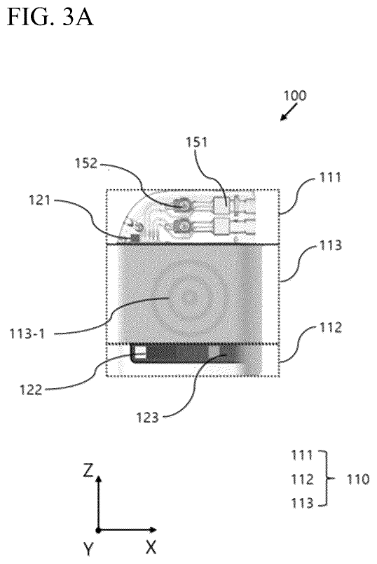

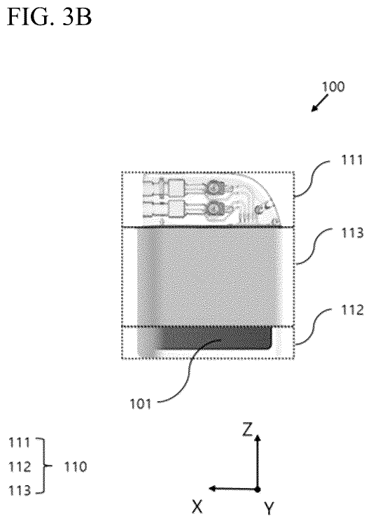

FIG. 3 is a front view and a rear view of the electroceutical.

Referring to FIG. 3A, a front view of the electroceutical 100 is shown, and referring to FIG. 3B, a rear view of the electroceutical 100 is shown.

The electroceutical 100 may include a housing 110 including a plurality of regions. The housing 110 may include a first silicon region 111, a second silicon region 112, and a titanium region 113.

The first silicon region 111 may be provided to cover at least a portion of a first antenna 121. The second silicon region 112 may be provided to cover a second antenna 122 and a third antenna 123. A plurality of the first to third antennas 121 to 123 operating in different frequency bands may be arranged on one surface of a printed circuit board (PCB) substrate 101.

A wireless communication signal may be transmitted or received through the first silicon region 111 and the second silicon region 112. At this time, the wireless communication signal may be attenuated by silicon. For example, in such a case that silicon having a Shore hardness of 60 A is included in the first silicon region 111 and the second silicon region 112, when a dielectric loss aϵᵣ is 3.7, a magnetic permeability is 4π×10⁻⁷ H/m, a frequency of the wireless communication signal is 2.4 GHz, and a thickness of the silicon is 5 to 15 mm, an attenuation α of the wireless communication signal may be approximately 0.00026 dB. That is, signal attenuation by the first silicon region 111 and the second silicon region 112 may be insignificant. The first silicon region 111 and the second silicon region 112 may preferably include silicon having a Shore hardness of 70 A or greater. When a Shore hardness of silicon is increased, transceiving sensitivity of a wireless communication signal may be increased.

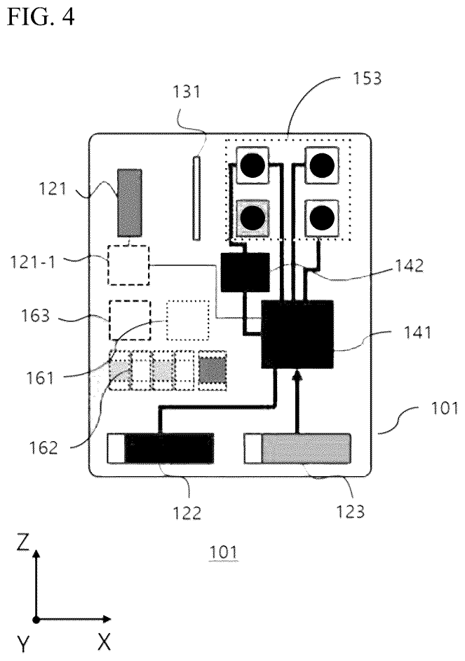

Additionally, the first silicon region 111 may cover a lead connector 151 and a screw 152. The lead connector 151 may be connected to a cuff lead (e.g., the cuff lead 12 of FIG. 2). The electroceutical 100 may sense an analog signal of a nerve or provide pulse stimulation to the nerve through the cuff lead connected to the lead connector 151. The screw 152 may be configured to connect the lead connector 151 to a header connector (e.g., a header connector 153 of FIG. 4).

The titanium region 113 may be located between the first silicon region 111 and the second silicon region 112. The titanium region 113 may be provided to cover the PCB substrate 101 and components placed on the PCB substrate 101. The titanium region 113 may include an ultrasound charging surface 113-1, and an ultrasound wave may be introduced into the electroceutical 100 through the ultrasound charging surface 113-1. Based on the ultrasound wave introduced into the electroceutical 100, a triboelectric power harvesting unit (e.g., the triboelectric power harvesting unit 200 of FIG. 8) may generate energy, and the generated energy may be used to operate the electroceutical 100. The triboelectric power harvesting unit is attached to an inner surface of the titanium region 113 to efficiently receive the ultrasound wave.

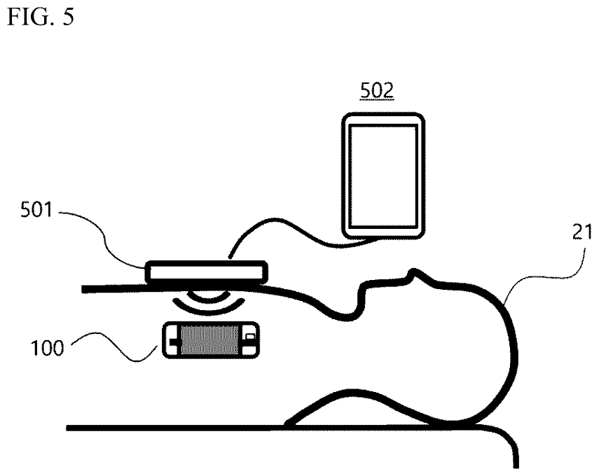

FIG. 4 is a front cross-sectional view of the electroceutical. FIG. 5 is a diagram for explaining a medical implant communication service (MICS).

Referring to FIG. 4, components arranged on the PCB substrate 101 may be identified. The plurality of first to third antennas 121 to 123 operating in different frequency bands may be arranged on the PCB substrate 101.

In a communications field, a method of using a frequency band of 400 MHz for a wake-up purpose and a frequency band of 2.4 GHz for a data communication purpose is being commercialized, instead of a method of using a frequency band of 2.4 GHz for a wake-up purpose and a frequency band of 400 MHz for a medical implant communication service (MICS). This is because Bluetooth communication in the 2.4 GHz frequency band may provide sufficient low power property, security, and reliability thanks to advancements in short-range communication technology, low-power communication semiconductor technology, and high reliability secured through process advancement.

However, the use of the frequency band of 2.4 GHz for data communication may improve directivity of a data communication signal, but may cause problems such as a decrease in transmittance of the data communication signal and occurrence of environmental noise interference. To solve these problems, there is a need for a specific method of arranging and controlling an antenna.

Power for the electroceutical 100 may be controlled through the first antenna 121 operating in a near-field communication (NFC) band. That is, the electroceutical 100 may receive a power control signal from outside through the first antenna 121 and control power through an NFC module 121-1 (e.g., turning on/off).

The first antenna 121 may be positioned adjacent to the header connector 153 compared to other antennas such as the second and third antennas 122 and 123. Since the first antenna 121 is configured to transceive a power control signal (an On/Off signal), relatively low reliability is needed, and since the first antenna 121 functions for inductance, an influence exerted on the header connector 153 may be small. However, even when the influence is small, noise may be present. Thus, shielding metal 131 (e.g., mu-metal including nickel, iron, copper, and/or molybdenum) may be arranged. The shielding metal 131 may extend in a longitudinal direction (e.g., a Z-axis direction) of the first antenna 121 and arranged between the first antenna 121 and the header connector 153. Additionally, to reduce noise, a capacitor of approximately 0.1 μF to 1 μF may be arranged between a power rail and ground by using a decoupling method.

In addition, the first antenna 121 has characteristics of a receiving antenna utilizing a short-range communication band to control power of the electroceutical 100, rather than utilizing a short-range communication band (e.g., an NFC band or a radio frequency identification (RFID) band) to charge the electroceutical 100 or transmit data. Thus, the first antenna 121 may have a small size compared to other antennas.

The second antenna 122 may have an operating frequency of 2.4 GHz. The second antenna 122 may transmit sensed biometric information to outside of the electroceutical 100 and receive a control signal for the electroceutical 100, the control signal being provided from outside.

The third antenna 123 may have an operating frequency of 400 or 900 MHz. The third antenna 123 may be configured for a medical implant communication service (MICS). In detail, the third antenna 123 may be configured for an emergency control/call according to safety.

Frequency bands allocated for emergency control functions vary depending on countries, but a frequency band of 402 to 405 MHz at which highest body transmission is shown is allocated in most countries. This frequency range is intended for use in communication services between implantable medical devices, and thus, may be used for devices implanted into a body for medical purposes (Class III) (e.g., the electroceutical 100).

Such implantable medical devices may use this frequency band to detect abnormalities present therein or to urgently provide control from outside. Due to high body transmissibility, the frequency band of 402 to 405 MHz is highly suitable for communications for medical use and providing low power characteristics, and may perform an important function for increasing reliability and safety of the implantable medical devices in emergency situations despite restrictions in use of frequencies.

Referring to FIG. 5, a method of providing communication services between implantable medical devices is shown. A communication pad 501 needs to be placed to be in close contact with a body of f the person 21 to perform communication services between the implantable medical devices. By doing so, a state of the electroceutical 100 may be monitored and the electroceutical 100 may be controlled as needed. For example, the electroceutical 100 may be controlled to notify a cell phone of the person 21 of a danger signal detected in the body of the person 21. Forced stop of the electroceutical 100 and emergency call control of the electroceutical 100 may be performed through the third antenna 123 configured for the MICS.

Although FIG. 5 shows a method of providing the MICS, the first antenna 121 utilizing an NFC frequency band (or a radio frequency identification (RFID) frequency band) may also function to control the electroceutical 100.

A MICS controls an electroceutical by employing an antenna device and components using frequencies that comply with national regulations. For example, when an electroceutical is urgently turned off via the MICS, a MICS-based RF chip needs to periodically wake up and scan an RF channel to turn the electroceutical on. However, periodic channel scanning consumes a lot of battery, and thus, may not be appropriate. Accordingly, power needs to be turned on using a method in which a battery is not consumed.

NFC may provide power using external radiation energy based on self-induction, and when used simply to perform a power control function, the NFC may be utilized even deep in a human body. Instead of NFC using a frequency of 13.56 MHz, a lower frequency band (e.g., 125 to 134 kHz) may also be used to perform simple power control.

When power is controlled through the NFC module 121-1, periodic energy consumption for MICS-based power control may not be needed. Power control using a traditional electromagnetic sensor may minimize a malfunction problem and reduce separate power consumption for a MICS-RF connection. Thus, this may help to operate the electroceutical 100 with a limited battery use.

The first antenna 121 connected to the NFC module 121-1 functions as a receiver (Rx), and is converted from a power-off state into a power-on state. Thus, the receiver (e.g., the first antenna 121) may have characteristics of an inductor configured to store energy of a magnetic field. Energy needed for the NFC module 121-1 performing power control may be sufficiently covered by only minimum energy stored in the first antenna 121.

That is, since the NFC module 121-1 receives power from the first antenna 121, a separate power line may not be needed. When power is supplied to the NFC module 121-1, an identification (ID) of a signal may be checked and, when matching the ID, a power control signal may be provided to a microcontroller unit (MCU) 141.

The MCU 141 may be a system on chip (SoC). The MCU 141 may perform sensing and stimulation simultaneously. Based on a control signal from an external electronic device, the MCU 141 may integrate and manage a function of an analog-to-digital converter (ADC) configured to sense an analog signal generated from a nerve and a function of pulse width modulation (PWM) for providing a stimulation to the nerve. By using the ADC function, the MCU 141 may sense an analog signal of 14 bits or more generated from the nerve with high resolution. By using the PWM function, the MCU 141 may provide the nerve with pulses of 0.1 Hz to several hundred kHz as a stimulation. The MCU 141 having integrated the functions may simplify an interface. To efficiently use the simplified interface (e.g., the header connector 153), the MCU 141 may change a setting of the header connector 153 according to an operating mode (e.g., a sensing mode or a stimulation mode).

The header connector 153 may include four header connectors. One of the four header connectors may be connected to ground to minimize unexpected errors (e.g., a sensing error or a stimulation error).

An operational (OP)-amplifier (AMP) 142 may be connected to the header connector 153. The OP-AMP 142 may be used to amplify or reduce a sensed signal or a provided stimulus (e.g., an electrical signal).

An alternating current (AC)-direct current (DC) converter 161 may convert AC triboelectricity generated by a triboelectric power harvesting unit (e.g., the triboelectric power harvesting unit 200 of FIG. 8) into DC triboelectricity.

A storage 162 may be a place for storing energy converted into DC (e.g., triboelectricity). The storage 162 may be implemented as a multi-stage capacitor (e.g., a system in which low resistance capacitors or supercapacitors of several hundred uF or several mF are connected to each other in series or parallel). The storage 162 may store energy in a wide frequency band (e.g., 0.2 to 200 kHz with a center frequency of 20 kHz). The storage 162 configured to store energy may also be provided to implement the electroceutical 100 free of a battery.

A power management integrated circuit (PMIC) 163 may be an integrated circuit configured to manage power for the electroceutical 100. The PMIC 163 may distribute power to elements included in the electroceutical 100. For example, the PMIC 163 may monitor a voltage to supply power to the MCU 141 or supply power to a battery (e.g., the battery 171 of FIG. 8) to charge the battery.

A battery management integrated circuit (BMIC) may be an integrated circuit configured to monitor and manage a state of the battery. The BMIC may manage charging and discharging of the battery. The BMIC and the battery may be placed on a rear surface (e.g., another surface) of the PCB substrate 101, or may not be included in the electroceutical 100.

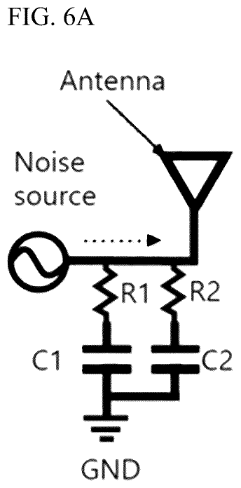

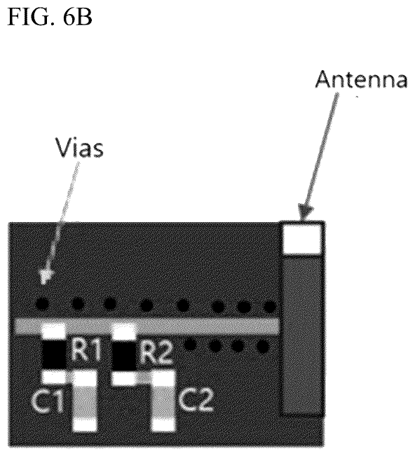

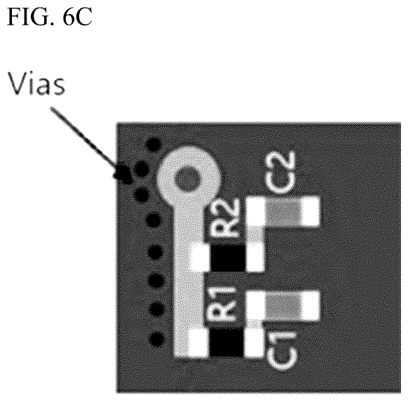

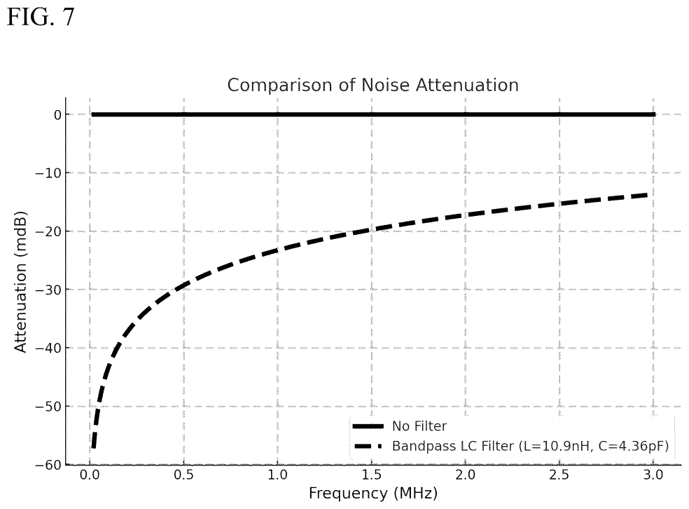

FIGS. 6 and 7 are diagrams for explaining noise filtering.

Referring to FIG. 6A and FIG. 6B, a circuit diagram of a noise filter connected to an antenna is shown. When an ultrasound wave for charging the electroceutical 100 is introduced into a housing, noise may occur. When ultrasound waves with frequencies of 20 kHz and 100 kHz are introduced, the noise filter connected to the antenna may have resistors of 1 kΩ (e.g., R1 and R2) and capacitors of 7.96 no and 1.59 nF (e.g., C1 and C2). Noise may be removed through vias configured to function for grounding.

Referring to FIG. 6C, a noise filter connected to a cuff lead is shown. Like the noise filter connected to the antenna, the noise filter connected to the cuff lead may minimize sensing errors during ultrasound charging by filtering and removing noise in bands of 19 to 21 kHz and 99 to 101 kHz.

However, although the noise filter may be configured as an RC filter in a low frequency range as shown in FIG. 6, an LC filter needs to be used in a high frequency range.

FIG. 7 is a graph showing a comparison of noise attenuations. Referring to FIG. 7, in such a case that a frequency range of an ultrasound wave for charging is 20 kHz to 3 MHz, noise attenuations 1) when a noise filter is not included and 2) when a noise filter including an inductor of 10.9 nH and a capacitor of 4.36 pF (e.g., an LC filter) are shown.

Referring to FIG. 7, it may be understood that a difference in noise attenuations depending on frequencies is present between when the noise filter is configured as a bandpass LC filter and when a noise filter is not included. In detail, when a frequency range of an ultrasound wave is 20 kHz, a noise attenuation of −40 dB (e.g., a noise reduction by about 1/100) may be obtained, and when a frequency range of an ultrasound wave is 3 MHz, a noise attenuation of −15 dB (e.g., noise improvement by approximately 15.85%) may be obtained.

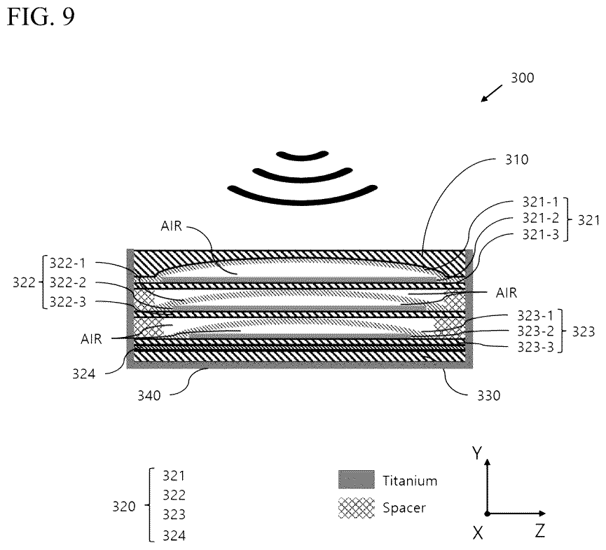

FIGS. 8 and 9 are diagrams for explaining a triboelectric power harvesting unit.

FIG. 8 is a right cross-sectional view of the electroceutical 100. Referring to FIG. 8, the electroceutical 100 (e.g., an electronic device) may include the triboelectric power harvesting unit 200 for charging. The triboelectric power harvesting unit 200 may be attached to a front surface (e.g., an inner surface of the titanium region 113 of the housing 110)) of the electroceutical 100 to effectively receive the ultrasound wave 24 provided from outside of the electroceutical 100.

The triboelectric power harvesting unit 200 may include a first silicon layer 210, a power generating unit 220, a second silicon layer 230, and a device housing 240. The triboelectric power harvesting unit 200 may be designed to 1) maximize charging efficiency, and 2) minimize an influence of the triboelectric power harvesting unit 200 on operation of the electroceutical 100 (e.g., sensing, stimulation, and communication).

The triboelectric power harvesting unit 200 may be attached to the inner surface of the titanium region 113 and spaced apart from the PCB substrate 101.

The first silicon layer 210 may be disposed between on the inner surface (e.g., a front surface) of the titanium region 113 and an upper surface of the power generating unit 220. The first silicon layer 210 may minimize an air gap between an inner surface of the electroceutical 100 (e.g., an inner surface made of titanium) and the power generating unit 220. Since it is difficult for an ultrasound wave to penetrate through the air gap, the first silicon layer 210 may be provided to prevent the ultrasound wave from failing to reach the power generating unit 220 and being reflected on the air gap (approximately 99% reflected). The first silicon layer 210 may maximize charging efficiency of the electroceutical 100 by increasing delivering efficiency of the ultrasound wave.

The power generating unit 220 may generate electricity based on an ultrasound wave (e.g., an ultrasound wave having penetrated through the housing 110 of the electroceutical 100 and the first silicon layer 210) provided from outside (e.g., outside the electroceutical 100). The power generating unit 220 may generate triboelectricity as internal components disposed therein vibrate due to the ultrasound wave. A configuration and operation of the power generating unit 220 will be described in detail with reference to FIG. 9.

The second silicon layer 230 may be disposed on a lower surface of the power generating unit 220. The second silicon layer 230 may suppress physical vibrations in units of μm generated in the power generating unit 220 from being delivered to outside of the triboelectric power harvesting unit 200. The second silicon layer 230 may be provided to minimize an influence of the triboelectric power harvesting unit 200 on operation of the electroceutical 100 (e.g., sensing, stimulation, or communication).

The device housing 240 may be arranged to surround outer peripheries of the first silicon layer 210, the power generating unit 220, and the second silicon layer 230. The device housing 240 may be also provided to minimize an influence of the triboelectric power harvesting unit 200 on operation of the electroceutical 100 (e.g., sensing, stimulation, or communication). The device housing 240 may be disposed to surround surfaces of the triboelectric power harvesting unit 200 other than an upper surface thereof to shield electromagnetic interference between the triboelectric power harvesting unit 200 and the outside. The device housing 240 may be made of mu-metal including nickel, iron, copper, and/or molybdenum to shield electromagnetic interference.

The mu-metal may effectively shield a magnetic field (e.g., 40 to 60 dB) in a low frequency band (e.g., a frequency band below 10 kHz) generated inside the triboelectric power harvesting unit 200. The triboelectric power harvesting unit 200 may also generate a magnetic field of several tens of mV while producing AC triboelectricity. The generated magnetic field may function as noise in other circuits.

Accordingly, the device housing 240 may be provided to surround surfaces of the triboelectric power harvesting unit 200 other than an upper surface thereof for receiving an ultrasound wave. The upper surface of the triboelectric power harvesting unit 200 may be attached to the inner surface of the electroceutical 100 (e.g., the inner surface of the titanium region 113) to package the triboelectric power harvesting unit 200. As a result, an operation error of the electroceutical 100 caused by the triboelectric power harvesting unit 200 may be minimized.

In addition, the mu-metal may effectively shield a magnetic field (e.g., a magnetic field introduced when magnetic resonance imaging (MRI) is performed on a body) introduced from outside of the triboelectric power harvesting unit 200.

FIG. 9 is a right side view of the triboelectric power harvesting unit 200.

Referring to FIG. 9, the right-side view of the triboelectric power harvesting unit 200 is shown. The triboelectric power harvesting unit 200 may include a first silicon layer 210, the power generating unit 220, the second silicon layer 230, and the device housing 240.

The power generating unit 220 may include a plurality of units 221 to 223 and a ceramic substrate 224. The plurality of units 221 to 223 may be stacked on the ceramic substrate 224.

The ceramic substrate 224 has a thermal conductivity of 200 W/mK which is 60 times higher than a thermal conductivity of a general PCB substrate (e.g., 0.4 W/mK), showing excellent heat dissipation performance. A thermal expansion coefficient of the ceramic substrate 224 is 7 ppm/° C. which is lower than a thermal expansion coefficient (e.g., 14 ppm/° C.) of a general PCB substrate (e.g., High Tg-PCB). The ceramic substrate 224 has a high temperature resistance of 800° C., which is much higher than a high temperature resistance (e.g., 185 to 220° C.) of a general PCB substrate (e.g., High Tg-PCB). Thus, the ceramic substrate 224 may withstand an extremely high temperature. In addition, the ceramic substrate 224 has a dielectric constant and a mechanical strength higher than those of a general PCB substrate (e.g., a flame retardant (FR)-4 PCB of 140 Mpa).

The plurality of units 221 to 223 stacked on the ceramic substrate 224 may include a first unit 221, a second unit 222, and a third unit 223.

The plurality of units 221 to 223 may include inductive bodies 221-1, 222-1, and 223-1 that vibrate according to ultrasound waves, charged bodies 221-2, 222-2, and 223-2 that generate triboelectricity according to friction with an inductive body, and supports 221-3, 222-3, and 223-3 each supporting a charged body, respectively.

The inductive bodies 221-1, 222-1, and 223-1 may include barium titanate (BaTiO₃). The inductive bodies 221-1, 222-1, and 223-1 may include thin films and vibrate. The inductive bodies 221-1, 222-1, and 223-1 may vibrate according to transmission of ultrasound waves. The inductive bodies 221-1, 222-1, and 223-1 may vibrate within the air layer AIR, and the inductive bodies 221-1, 222-1, and 223-1 may be provided to have a thickness of 15 μm or less to efficiently vibrate.

The charged bodies 221-2, 222-2, and 223-2 may contain gold (Au). The charged bodies 221-2, 222-2, and 223-2 may generate triboelectricity due to friction with a vibrating inductive body. The charged bodies 221-2, 222-2, and 223-2 may contain not only gold (Au), but also a material in which frictional electricity (e.g., nickel, silver, etc.) may be easily generated.

The supports 221-3, 222-3, and 223-3 may support a charged body. Spacers may be interposed between the supports 221-3, 222-3, and 223-3. Areas of respective units may be distinguished from each other by the spacers.

In the first unit 221 and the second unit 222, the supports 221-3 and 222-3 may include barium titanate, zirconia (Zro₂), and/or alumina (Al₂O₃). Zirconia (ZrO₂) and alumina (Al₂O₃) may be materials having high reflectivity. Zirconia (ZrO₂) and alumina (Al₂O₃) may have a reflectivity 10 times higher than that of FR-4 including an epoxy resin and glass fiber.

For example, in the first unit 221, an ultrasound wave having penetrated through the inductive body 221-1, the air layer AIR, and the charged body 221-2 may be reflected by the support 221-3 with high reflectivity. The reflected ultrasound wave may induce repetitive vibrations within the first unit 221. Repetitive vibrations may increase energy efficiency of the triboelectric power harvesting unit 200. That is, an ultrasound wave provided from outside may penetrate through the titanium region 113 and the first silicon layer 210 to be introduced into the power generating unit 220, and reflected in the power generating unit 220 to cause repetitive vibrations.

In the third unit 223, the support 223-3 may include a fluorine compound (e.g., perfluoroalkoxy (PFA), fluorinated tetrafluoroethylene (FTFE), perfluorodecanoic acid (PFDA), and polydiacetylene (PDA)). The fluorine compound constituting the support 223-3 may have a higher reflectivity than that of zirconia (ZrO₂) and alumina (Al₂O₃) constituting the supports 221-3 and 222-3. Referring to Table 1, a transmittance of each material is shown (transmittance=1−reflectivity).

TABLE 1

| Transmittance | Al²O³ | BTO | ZrO² | FR-4 | PDA | PFA | PVDF | PFDA | PTFE |
|---|---|---|---|---|---|---|---|---|---|
| Alumina (Al²O³) | 0.074 | 0.068 | 0.066 | 0.016 | 0.011 | 0.010 | 0.015 | 0.010 | 0.010 |
| BTO (BaTiO³) | 0.068 | 0.062 | 0.061 | 0.015 | 0.010 | 0.009 | 0.014 | 0.009 | 0.009 |
| Zirconia (ZrO²) | 0.066 | 0.061 | 0.060 | 0.015 | 0.009 | 0.009 | 0.013 | 0.009 | 0.009 |
| FR-4 | 0.016 | 0.015 | 0.015 | 0.004 | 0.002 | 0.002 | 0.004 | 0.002 | 0.002 |
| PDA(Polydopamine) | 0.011 | 0.010 | 0.009 | 0.002 | 0.001 | 0.001 | 0.002 | 0.001 | 0.001 |
| PFA (Perfluoroalkoxy) | 0.010 | 0.009 | 0.009 | 0.002 | 0.001 | 0.001 | 0.002 | 0.001 | 0.001 |
| PVDF | 0.015 | 0.014 | 0.013 | 0.004 | 0.002 | 0.002 | 0.004 | 0.002 | 0.002 |
| (Polyvinylidene fluoride) | | | | | | | | | |
| PFDA | 0.010 | 0.009 | 0.000 | 0.002 | 0.001 | 0.001 | 0.002 | 0.001 | 0.001 |
| (Perfluorodecanoic acid) | | | | | | | | | |
| PTFE | 0.010 | 0.009 | 0.009 | 0.002 | 0.001 | 0.001 | 0.002 | 0.001 | 0.001 |
| (Polytetrafluoroethylene) | | | | | | | | | |

An ultrasound wave having penetrated through the first unit 221, the second unit 222, the inductive body 223-1, the air layer, and the charged body 223-2 may be reflected by the support 223-3 with very high reflectivity. The reflected ultrasound wave may induce additional vibrations within the triboelectric power harvesting unit 200. The additional vibrations may further increase energy efficiency of the triboelectric power harvesting unit 200.

Referring to Table 1 and FIG. 9, the inductive body 221-1 of the first unit 221 is in contact with the first silicon layer 210, and thus, may include barium titanate (BaTiO₃) having a highest transmittance with respect to silicon. In addition, the supports 221-3 and 222-3 in the first unit 221 and the second unit 222 may include alumina (Al₂O₃) and/or zirconia (ZrO₂) having relatively high transmittance with respect to gold (Au), and the support 223-2 of the third unit 223 may include a fluorine compound (e.g., PFA, FTFE, PFDA, or PDA) having a relatively low transmittance with respect to gold (Au). A power generation amount of the triboelectric power harvesting unit 200 may be improved by configuring a material of each structure in consideration of a transmittance and an amount of reflection of an ultrasound wave.

Although the present disclosure has been described with reference to an embodiment illustrated in the drawings, this is only an example, and it will be understood by those of ordinary skill in the art that various changes in the form and details may be made therein without departing from the spirit and scope of the present disclosure.
This research patent was supported by the Ministry of SMEs and Startups and the Korea Startup Promotion Agency through the DIPS 1000+ program (20241755) as part of the Super Gap Startup Development Project.
This research patent was supported by the 2022 research fund from the Ministry of Science and ICT and the National Research Foundation of Korea (NRF) under the Electronic Medicine Technology Development Program (2022M3E5E9016662).
This research patent was supported by the 2023 research fund from the Ministry of Trade, Industry and Energy and the Korea Evaluation Institute of Industrial Technology (KEIT) under the Next-Generation Intelligent Semiconductor Technology Development Program (20025736).

1. An electroceutical comprising:
a housing comprising a plurality of regions;
a triboelectric power harvesting unit configured to generate energy based on an ultrasound wave provided from outside of the electroceutical;
a plurality of antennas operating in different frequency bands; and
shielding metal configured to shield electromagnetic interference by at least one of the plurality of antennas,
wherein the plurality of antennas comprise:
a first antenna configured to transceive a power control signal for the electroceutical through a near field communication (NFC) band;
a second antenna having an operating frequency of 2.4 GHz; and
a third antenna configured for a medical implant communication service (MICS),
wherein the plurality of regions in the housing comprise:
a first silicon region provided to cover at least a portion of the first antenna;
a second silicon region provided to cover the second antenna and the third antenna; and
a titanium region located between the first silicon region and the second silicon region,
further comprising a printed circuit board (PCB) substrate,
wherein the plurality of antennas and the shielding metal are arranged on one surface of the PCB substrate, and
the triboelectric power harvesting unit is attached to an inner surface of the titanium region and is spaced apart from the PCB substrate,
further comprising a header connector arranged on the one surface of the PCB substrate,
wherein the first antenna is arranged further adjacent to the header connector compared to the second antenna and the third antenna, and
the shielding metal extends in a longitudinal direction of the first antenna to be arranged between the first antenna and the header connector.

2.-5. (canceled)

6. The electroceutical of claim 1, further comprising a microcontroller unit (MCU) configured to change a setting of the header connector according to an operation mode,
wherein, based on a control signal from an external electronic device, the MCU integrates and manages a function of an analog-to-digital converter (ADC) configured to sense an analog signal generated from a nerve and a function of a pulse width modulation (PWM) configured to provide a stimulation to the nerve.

7. The electroceutical of claim 6, further comprising:
an ADC configured to convert alternating current (AC) triboelectricity generated by the triboelectric power harvesting unit into direct current (DC) triboelectricity; and
a storage configured to store the DC triboelectricity obtained as a result of the converting.

8. The electroceutical of claim 7, wherein the triboelectric power harvesting unit comprises:
a power generating unit comprising a first unit, a second unit, and a third unit;
a first silicon layer arranged between an upper surface of the power generating unit and an inner surface of the electroceutical;
a second silicon layer arranged on a lower surface of the power generating unit; and
a device housing arranged to surround outer peripheries of the power generating unit, the first silicon layer, and the second silicon layer.

9. The electroceutical of claim 8, wherein the ultrasound wave provided from the outside penetrates through a titanium region and the first silicon layer to be introduced into the power generating unit, is reflected within the power generating unit, and causes repetitive vibrations.

10. The electroceutical of claim 9, wherein the device housing and the shielding metal comprise mu-metal comprising at least one among nickel, iron, copper, and molybdenum.
